(19) United States
(12) Patent Application Publication
(10) Pub. No.: US 2014/0021047 A1
(43) Pub. Date: Jan. 23, 2014

(54) METHOD FOR ANALYZING BIOMOLECULES USING ASYMMETRIC ELECTROLYTE CONCENTRATION

(71) Applicant: Samsung Electronics Co., Ltd., (US)

(72) Inventors: Jeo-young SHIM, Yongin-si (KR); Dong-ho LEE, Seongnam-si (KR); Kun-sun EOM, Seoul (KR); Tae-han JEON, Hwaseong-si (KR); Hee-jeong JEONG, Seoul (KR)

(73) Assignee: SAMSUNG ELECTRONICS CO., LTD., Suwon-si (KR)

(21) Appl. No.: 13/781,343

(22) Filed: Feb. 28, 2013

(30) Foreign Application Priority Data
Jul. 20, 2012 (KR) 10-2012-0079586

(51) Int. Cl. G01N 27/447; B01L 3/00

(52) U.S. Cl. CPC G01N 27/44791; B01L 3/50273; B82Y 5/00
USPC 204/451; 204/601; 977/962; 977/781

(57) ABSTRACT

A method and system for analyzing biomolecules using a high concentration electrolytic solution and a low concentration electrolytic solution.

### CROSS-REFERENCE TO RELATED APPLICATIONS

This application claims the benefit of Korean Patent Application No. 10-2012-0079586, filed on Jul. 20, 2012, in the Korean Intellectual Property Office, the disclosure of which is incorporated herein in its entirety by reference.

### BACKGROUND

1. Field

The present disclosure relates to methods and systems for analyzing biomolecules using a nanopore device.

A. 2. Description of the Related Art

Methods for detecting target biomolecules in a sample have been developed. Among these methods, research into a method using a nanogap has been conducted as a DNA detection system that mimics a biopore. For example, a tunneling current or a blockade current can be measured while DNA or RNA passes through a nanogap.

However, if an electrode used to measure the current is not insulated, or the concentration of a salt of a medium used in the measurement is high, significant noise is generated in measured signals. In addition, if a medium with a low salt concentration is used, it is difficult to analyze long biomolecules since folding of biomolecules, such as proteins and nucleic acids, cannot be effectively inhibited.

Thus, there is a need to develop new methods of analyzing biomolecules using a nanopore or nanogap.

### SUMMARY

Provided is a method of analyzing biomolecules by providing a first electrolytic solution containing biomolecules to a cis chamber of a device that comprises a cis chamber for holding a liquid; a trans chamber for holding a liquid; a substrate comprising one or more nanopores that penetrate the substrate in a thickness direction, wherein the nanopores have a first end and a second end opposite to the first end which are in fluid communication with the cis chamber and the trans chamber, respectively; and one or more electrodes positioned to apply a voltage to a liquid that passes through the one or more nanopores. The method further comprises providing a second electrolytic solution to the trans chamber; translocating the biomolecules from the cis chamber to the trans chamber; and measuring an electric signal that is caused by the translocation of the biomolecules through the one or more nanopores, wherein a ratio of a concentration of electrolyte in the first electrolytic solution to a concentration of electrolyte in the second electrolytic solution is equal to or greater than 10:1.

Also provided is a system for analyzing biomolecules, the system comprising a cis chamber containing a first electrolytic solution comprising biomolecules; a trans chamber containing a second electrolytic solution; a substrate comprising one or more nanopores that penetrate the substrate in a thickness direction and have a first end and a second end opposite to the first end which are in fluid communication with the cis chamber and the trans chamber, respectively; and a pair of electrodes positioned to apply a voltage to a liquid that passes through the one or more nanopores; wherein the pair of electrodes comprises a first electrode and a second electrode disposed on the substrate, wherein the first electrode and the second electrode define at least a portion of the wall of the one or more nanopores such that the first and second electrodes are in contact with the internal space of the one or more nanopores; and wherein a ratio of a concentration of electrolyte in the first electrolytic solution to that in the second electrolytic solution is equal to or greater than 10:1.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic view of a device for linearly translocating biomolecules via pores;

FIG. 2 is an enlarged view of the portion which defines a nanopore of a substrate of the FIG. 1;

FIG. 3 is a diagram illustrating a method of manufacturing the device of FIG. 1;

FIG. 4 is a schematic diagram illustrating a device for measuring leakage current of an insulating layer of FIG. 4;

FIG. 5 is a schematic diagram illustrating a device for observing changes of noise and a power spectrum according to the concentration of electrolyte;

FIG. 6 is a graph illustrating noise measured according to the concentration of the electrolyte;

FIGS. 7A and 7B are graphs of current plotted against time illustrating noise of distilled water and 1 M KCl;

FIG. 8 is a graph of current measured with respect to DNA transit time; and

FIGS. 9 to 11 are graphs of current plotted against translocation time illustrating changes of current with respect to DNA transit time determined in FIG. 8.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

According to an aspect of the present invention, a method of analyzing biomolecules includes: providing the first electrolytic solution containing biomolecules to a cis chamber of a device that includes: the cis chamber for holding a liquid; a trans chamber for holding a liquid; a substrate including one or more nanopores that penetrate the substrate in a thickness direction, wherein the nanopore have a first end and a second end opposite to the first end which are in fluid communication with the cis chamber and the trans chamber, respectively; and one or more electrodes positioned to apply a voltage to a liquid that passes through the nanopores; providing a second electrolytic solution to the trans chamber; translocating the biomolecules from the cis chamber to the trans chamber; and measuring an electric signal that is caused by the translocation of the biomolecules through the nanopores, wherein a ratio of a concentration of an electrolyte in the first electrolytic solution to a concentration of an electrolyte in the second electrolytic solution is equal to or greater than 10:1.

The concentration of an electrolyte in the first electrolytic solution may be any suitable concentration, such as 1 M or less, for example, 0.8 M or less, 0.5 M or less, or 0.2 M or less. The concentration of an electrolyte in the first electrolytic solution may be in a range of 0.1 mM to 1 M, for example, 0.1 mM to 0.8 M, 0.1 mM to 0.5 M, or 0.1 mM to 0.2 M. The concentration of an electrolyte in the second electrolytic solution may be any concentration sufficient to provide a ratio of concentration in the first electrolyte solution to that of the second equal to or greater than 10:1. For instance, the concentration of electrolyte in the second electrolyte solution may be 100 mM or less, for example, 50 mM or less, 30 mM or less, 10 mM or less, 1 mM or less, 800 μM or less, 500 μM or less, 300 μM or less, 100 μM or less, 50 μM or less, 10 μM or less, 5 μM or less, or 1 μM or less. The concentration of an electrolyte in the second electrolytic solution may be in a range of 0.1 μM to 100 mM, for example, 0.1 μM to 50 mM, 0.1 μM to 30 mM, 0.1 μM to 10 mM, 0.1 μM to 1 mM, 0.1 μM to 800 μM, 0.1 μM to 500 μM, 0.1 μM to 300 μM, 0.1 μM to 100 μM, 0.1 μM to 50 μM, 0.1 μM to 10 μM, 0.1 μM to 5 μM, or 0.1 μM to 1 μM.

Used herein, the term “electrolyte” refers to a material that has free ions that make the material be electrically conductive. Examples of the electrolyte include acids, bases, salts, or a combination thereof. The term “salts” includes ionic compounds generated by the neutralization reaction between acids and bases. The salts consisting of cations and anions are electrically neutral. Molten salts and solutions including salts dissolved in water, such as a NaCl solution, are referred to as electrolytes, and the electrolytes have electrical conductivity. The pH of acidic or basic solutions may be in a range of about 4 to about 9. The acid may be an organic or inorganic acid. The organic acid may include formic acid, acetic acid, lactic acid, citric acid, oxalic acid, or any combination thereof. The inorganic acid may include hydrochloric acid, nitric acid, phosphoric acid, sulfuric acid, boric acid, or any combination thereof. The base may be an organic or inorganic base. The organic base may include pyridine, methyl amine, imidazole, histidine, or any combination thereof. The inorganic base may include ammonia, ammonium hydroxide, ammonium carbonate, or any combination thereof.

The method includes providing a first electrolytic solution containing biomolecules to a cis chamber of a device that includes: the cis chamber for holding a liquid; a trans chamber for holding a liquid; a substrate including nanopores that are connected to the cis chamber and the trans chamber in a fluid communicable manner, wherein the nanopores are formed to penetrate the substrate in a thickness direction and have a first end and a second end opposite to the first end which are respectively connected to the cis chamber and the trans chamber; and an electrode that is disposed to apply a voltage to a liquid that passes through the nanopores.

The cis and trans chambers may be any structure suitable for holding a liquid. For example, the chambers may have a sealed structure with an entrance (inlet), the opening and closing of which are controlled, or a structure one portion of which is open. The cis and trans chambers may be temporarily or permanently sealed or sealable after introduction of the electrolyte solution.

The term “biomolecule” includes bio-derived polymers (biopolymers). The biomolecule may be, for example, nucleic acid, protein, sugar including polysaccharide, or any combination thereof. In some embodiments, the biomolecules may be nucleic acids, such as DNA, RNA, or any combination thereof. The nucleic acid may be selected from a single-stranded nucleic acid, a double-stranded nucleic acid, or any combination thereof. The nucleic acid may have a secondary or tertiary structure.

The substrate includes nanopores that are connected to the cis chamber and the trans chamber in a fluid communicable manner. The nanopores are formed to penetrate the substrate in a thickness direction, and have a first end and a second end opposite to the first end, which are respectively connected to the cis chamber and the trans chamber. The nanopore provides a path through which a fluid flows. The nanopore may connect the first end and the second end in a linear or curved manner to allow the fluid to flow therebetween. The substrate may be a solid substrate that may support the flow of the fluid.

The substrate may include a non-biological material rather than a bio-derived material such as a biomembrane. The substrate may include an insulating material. The substrate may include silicon nitride (Si₃N₄), aluminum oxide (Al₂O₃), silica (SiO₂), a plastic such as Teflon™, an elastomer such as a two-component addition cured silicone rubber, and any combination thereof. The substrate may have a flat shape, for example, a film or a layer shape or an irregular shape. The substrate may have a shape in which at least one portion in contact with the first end of the nanopore is flat (e.g., at least one side of the substrate in a region where the nanopore is formed is flat). The substrate may have a layered structure in which a thin film such as a silicon thin film is supported by a support material. The thickness of the portion where the nanopores are disposed may be in a range of about 1 nm to about 1000 nm, for example, about 3.4 nm to about 500 nm or about 1 nm to about 50 nm.

The length of a cross-section of the nanopore may be in a range of 1 nm to 100 nm, for example, 1 nm to 5 nm, 1 nm to 10 nm, 5 nm to 10 nm, or 1 nm to 25 nm. The cross-section of the nanopore may have a circular or polygonal shape. If the nanopore has a circular cross-section, the length of the cross-section indicates a diameter. If the nanopore has a polygonal cross-section, the length of the cross-section indicates the shortest length. The length of the cross-section of the nanopore may be uniform in the longitudinal direction of the nanopore. The longitudinal length of the nanopore is not particularly limited as long as a biomolecule passes therethrough. The longitudinal length of the nanopore may be less than the length of the biomolecule passing through the nanopore. The longitudinal length of the nanopore may be longer than a distance between bases of the biomolecule, for example, in a range of 3.4 nm to 500 nm. In addition, the longitudinal length of the nanopore may be equal or shorter than a distance between bases of the biomolecule, for example, 3.4 nm or less.

The device includes one or more electrodes disposed to apply a voltage to a liquid that flows through the nanopore. The electrode may be provided by a pair of electrodes including a first electrode and a second electrode disposed on the substrate, wherein the first electrode and the second electrode define at least a part of walls of the nanopores so as to be in contact with the internal space of the nanopore. The pair of the first electrode and second electrode is electrically connected to a power source and/or an electric signal measuring device. The first and second electrodes are disposed to be spaced apart from each other by an insulating material.

The device may further include a third electrode that is disposed at the first end side of the nanopore and a fourth electrode that is disposed at the second end side of the nanopore. In other words, the third and fourth electrodes may be positioned opposite one another relative to the nanopore, e.g., on opposite sides of the substrate comprising the nanopore. For instance, the third electrode may be positioned at or in the vicinity of a first end of the nanopore and the fourth electrode may be positioned at or in the vicinity of a second end of the nanopore. The electrodes may be positioned within the nanopore, on the substrate at the opening to the nanopore, or in the cis and trans chambers. The third electrode and the fourth electrode may be electrically connected to a voltage source and/or an electric signal measuring device.

In the device, insulating layers may be disposed on the upper and lower surfaces of the first electrode and the second electrode, and thus the upper and lower surfaces are insulated from the first electrolytic solution and the second electrolytic solution. The insulating layer may include silicon nitride, silicon oxide, aluminum oxide, hafnium oxide, or any combination thereof.

The device may further include a translocation unit that linearly translocates the biomolecule, for example, a biological polymer, through the nanopore. The translocation unit may be a unit for providing a concentration gradient, voltage gradient, or magnetic force gradient (or any combination thereof) between the first and second ends of the nanopore. The translocation unit may include, for example, at least two electrodes disposed so as to apply a voltage between the first and second ends. The electrode may define at least one portion of the nanopore or may be separated from the nanopore. If the electrode defines at least one portion of the nanopore, at least one portion of the nanopore may be formed of a conductive material. For example, at least one portion of the nanopore may be coated or impregnated with a conductive material. The translocation unit may include a molecular motor, a mechanical drive device, or any combination thereof disposed between the first and second ends or disposed in the channel. The device may include a power source that is electrically connected to the translocation unit.

In the device, the cis chamber may be a chamber for amplifying nucleic acids or may be connected to the chamber for amplifying nucleic acids in a fluid communicable manner. In addition, the cis chamber may be connected to a reservoir for storing a reagent or a material in a fluid communicable manner.

The providing of the first electrolytic solution containing biomolecules to the cis chamber may be conducted by distributing the first electrolytic solution into the cis chamber manually or using a mechanical unit, for example, a pump. The biomolecules may be nucleic acids. The nucleic acid may be DNA, RNA, or any combination thereof. The nucleic acid may be selected from a single-stranded nucleic acid, a double-stranded nucleic acid, and any combination thereof. The nucleic acid may have a secondary or tertiary structure. Biomolecules, for example, nucleic acids are not combined with other polymers but separated from other polymers. The nucleic acid may be an amplified product. The first electrolytic solution may have a salt concentration in a range of 1 mM to 1 M. The first electrolytic solution and the second electrolytic solution may include the same type of salts. The salt may include KCl, NaCl, LiCl, or any combination thereof. The first electrolytic solution and/or the second electrolytic solution may be an acidic or basic solution. In this regard, the pH of the first electrolytic solution and/or the second electrolytic solution may be in a range of about 4 to about 9.

The method includes providing the second electrolytic solution to the trans chamber. The providing of the second electrolytic solution to the trans chamber may be conducted by distributing the second electrolytic solution into the trans chamber manually or using a mechanical unit, for example, a pump. The second electrolytic solution may include a salt with a concentration in a range of 0.1 μM to 0.1 M. The salt may include KCl, NaCl, LiCl, or any combination thereof.

The concentration ratio of the electrolyte between the first electrolytic solution and the second electrolytic solution may be 10:1 or greater, for example, in a range of 10 to 20:1, 10 to 50:1, 10 to 100:1, 20 to 100:1, 50 to 100:1, 80 to 100:1, or 10 to 100,000:1.

The method includes translocating the biomolecules from the cis chamber to the trans chamber. The translocating may be conducted by any driving force applied to the biomolecules across or between the first and second ends. The translocation may be conducted by applying natural gravity, diffusion, voltage gradient, magnetic force gradient, molecular motor, mechanical power, or any combination thereof to the biomolecules. For example, a voltage gradient may be applied across the first and second ends of the nanopore, which are in contact with an electrolytic solution. The electrolytic solution may be any solution including, for example, KCl, NaCl, LiCl, or any combination thereof.

The method includes measuring an electric signal that is caused by the translocation of the biomolecules through the nanopores. The translocation may be a linear translocation via the nanopore. The electric signal may be generated by changes of electrical characteristics caused by the linear translocation of the biomolecules via the nanopores. The characteristics include electrical characteristics such as current and voltage. For example, the changes of the electrical characteristics may include the degree of reduction or increase in current caused while the biomolecules are linearly translocated through the nanopores and a time period during which the changes occur. That is, the changes of electrical characteristics with time are measured, and the translocation of the biomolecules caused by the changes is measured. The measuring may be conducted by measuring current change when both of the first end and the second end contact the electrolytic solutions.

The measuring may be conducted by measuring a tunneling current between the biomolecule passing through the nanopore and the electrode, or a blockade current caused by the biomolecule passing through nanopore by using the electric signal measuring device. The measuring may be conducted by measuring an increase of current between the biomolecule passing through the nanopore and the electrode by using the electric signal measuring device.

According to an embodiment, the first electrolytic solution and the second electrolytic solution may only include a salt, acid, or base.

The method may further include determining a sequence of the biomolecule based on the measured electric signal. The method may further include determining the presence or concentration of a sequence of the biomolecule based on the measured electric signal.

According to another aspect of the present invention, a device or system including the device for analyzing biomolecules includes: a cis chamber that holds a first electrolytic solution including biomolecules; a trans chamber that holds a second electrolytic solution; a substrate including nanopores that are connected to the cis chamber and the trans chamber in a fluid communicable manner, wherein the nanopores are formed to penetrate the substrate in a thickness direction and have a first end and a second end opposite to the first end which are respectively connected to the cis chamber and the trans chamber; and an electrode that is disposed to apply a voltage to a liquid that passes through the nanopores, wherein the electrode includes a pair of a first electrode and a second electrode disposed on the substrate, wherein the first electrode and the second electrode define at least a portion of the walls of the nanopores between the first and second electrodes to be in contact with the internal space of the nanopore, wherein a ratio of a concentration of an electrolyte in the first electrolytic solution to that in the second electrolytic solution is equal to or greater than 10:1.

The concentration of an electrolyte in the first electrolytic solution may be any suitable concentration, such as 1 M or less, for example, 0.8 M or less, 0.5 M or less, or 0.2 M or less. The concentration of the electrolyte in the first electrolytic solution may be in a range of 0.1 mM to 1 M, for example, 0.1 mM to 0.8 M, 0.1 mM to 0.5 M, or 0.1 mM to 0.2 M. The concentration of an electrolyte in the second electrolytic solution may be any concentration sufficient to provide a ratio of concentration in the first electrolyte solution to that of the second equal to or greater than 10:1. For instance, the concentration of electrolyte in the second electrolyte solution may be 100 mM or less, for example, 50 mM or less, 30 mM or less, 10 mM or less, 1 mM or less, 800 μM or less, 500 μM or less, 300 μM or less, 100 μM or less, 50 μM or less, 10 μM or less, 5 μM or less, or 1 μM or less. The concentration of the electrolyte in the second electrolytic solution may be in a range of 0.1 μM to 100 mM, for example, 0.1 μM to 50 mM, 0.1 μM to 30 mM, 0.1 μM to 10 mM, 0.1 μM to 1 mM, 0.1 μM to 800 μM, 0.1 μM to 500 μM, 0.1 μM to 300 μM, 0.1 μM to 100 μM, 0.1 μM to 50 μM, 0.1 μM to 10 μM, 0.1 μM to 5 μM, or 0.1 μM to 1 μM. The first electrolytic solution and the second electrolytic solution may include the same type of salts. The salt may include KCl, NaCl, LiCl, or any combination thereof. The first electrolytic solution and/or the second electrolytic solution may be an acidic or basic solution. In this regard, the pH of the first electrolytic solution and/or the second electrolytic solution may be in a range of 4 to 9. The second electrolytic solution may include a salt with a concentration in a range of 0.1 μM to 0.1 M. The salt may include KCl, NaCl, LiCl, or any combination thereof.

The concentration ratio of the electrolyte between the first electrolytic solution and the second electrolytic solution may be 10:1 or greater, for example, in a range of 10 to 20:1, 10 to 50:1, 10 to 100:1, 20 to 100:1, 50 to 100:1, 80 to 100:1, or 10 to 100,000:1.

The cis and trans chambers may by any structure suitable for holding a liquid. For example, the chambers may have a sealed structure with an entrance (inlet), the opening and closing of which are controlled, or a structure, one portion of which is open. The cis and trans chambers may be temporarily or permanently sealed or sealable after introduction of the electrolyte solution.

The biomolecules may be any biomolecule as described herein with respect to the method of analyzing a biomolecule. The substrate includes nanopores that are connected to the cis chamber and the trans chamber in a fluid communicable manner. The nanopores are formed to penetrate the substrate in a thickness direction and have a first end and a second end opposite to the first end which are respectively connected to the cis chamber and the trans chamber. The nanopore provides a path through which the fluid flows. The nanopore may connect the first end and the second end in a linear or curved manner to allow the fluid to flow therebetween. The substrate may be a solid substrate that may support the flow of the fluid.

The substrate may include a non-biological material rather than a bio-derived material such as a biomembrane. The substrate may include an insulating material. The substrate may include silicon nitride (Si₃N₄), aluminum oxide (Al₂O₃), silica (SiO₂), a plastic such as Teflon™, an elastomer such as a two-component addition cured silicone rubber, and any combination thereof. The substrate may have a flat shape, for example, a film or a layered shape or an irregular shape. The substrate may have a shape in which at least one portion in contact with the first end of the nanopore is flat (e.g., at least one side of the substrate in a region where the nanopore is formed is flat). The substrate may have a layered structure in which a thin film such as a silicon thin film is supported by a support material. The thickness of the portion where the nanopores are disposed may be in a range of 1 nm to 1000 nm, for example, 3.4 nm to 500 nm or 1 nm to 50 nm.

The length of a cross-section of the nanopore may be in a range of 1 nm to 100 nm, for example, 1 nm to 5 nm, 1 nm to 10 nm, 5 nm to 10 nm, or 1 nm to 25 nm. The cross-section of the nanopore may have a circular or polygonal shape. If the nanopore has a circular cross-section, the length of the cross-section indicates a diameter. If the nanopore has a polygonal cross-section, the length of the cross-section indicates the shortest length. The length of the cross-section of the nanopore may be uniform in the longitudinal direction of the nanopore. The longitudinal length of the nanopore is not particularly limited as long as a biomolecule passes therethrough. The longitudinal length of the nanopore may be less than the length of the biomolecule passing through the nanopore. The longitudinal length of the nanopore may be equal or longer than a distance between bases of the biomolecule, for example, in a range of 3.4 nm to 500 nm. In addition, the longitudinal length of the nanopore may be equal or shorter than a distance between bases of the biomolecule, for example, 3.4 nm or less.

The device includes one or more electrodes disposed to apply a voltage to a liquid that flows through the nanopore. The electrode may be provided by a pair of electrodes including a first electrode and a second electrode disposed on the substrate, wherein the first electrode and the second electrode define at least a part of walls of the nanopores so as to be in contact with the internal space of the nanopore. The pair of the first and second electrodes is electrically connected to a power source and/or an electric signal measuring device. The first and second electrodes are disposed to be spaced apart from each other by an insulating material.

The device may further include a third electrode that is disposed at the first end side of the nanopore and a fourth electrode that is disposed at the second end side of the nanopore. In other words, the third and fourth electrodes may be positioned opposite one another relative to the nanopore, e.g., on opposite sides of the substrate comprising the nanopore. For instance, the third electrode may be positioned at or in the vicinity of a first end of the nanopore and the fourth electrode may be positioned at or in the vicinity of a second end of the nanopore. The electrodes may be positioned within the nanopore, on the substrate at the opening to the nanopore, or in the cis and trans chambers. The third electrode and the fourth electrode may be electrically connected to a voltage source and/or an electric signal measuring device. The third and fourth electrodes may be used to provide electrical driving force to translocate the biomolecules via the nanopores.

In the device, insulating layers may be disposed on the upper and lower surfaces of the first electrode and the second electrode, and thus the upper and lower surfaces are insulated from the first electrolytic solution and the second electrolytic solution. Thus, the first and second electrodes only contact the portion of electrolytic solution that may be in the nanopore. The insulating layer may include silicon nitride, silicon oxide, aluminum oxide, hafnium oxide, or any combination thereof.

The device may further include a translocation unit that linearly translocates the biomolecule, for example, a biological polymer, through the nanopore. The translocation unit may be a unit for providing a concentration gradient, voltage gradient, and magnetic force gradient (or combination thereof) between the first and second ends of the nanopore. The translocation unit may include, for example, at least two electrodes disposed so as to apply a voltage between the first and second ends or disposed in a channel. The electrode may define at least one portion of the nanopore or may be separated from the nanopore. If the electrode defines at least one portion of the nanopore, at least one portion of the nanopore may be formed of a conductive material. For example, at least one portion of the nanopore may be coated or impregnated with a conductive material. The translocation unit may include a molecular motor, a mechanical drive device, or any combination thereof disposed between the first and second ends or in the channel. The device may include a power source that is electrically connected to the translocation unit.

In the device, the cis chamber may be a chamber for amplifying nucleic acids or may be connected to the chamber for amplifying nucleic acids in a fluid communicable manner. In addition, the cis chamber may be connected to a reservoir for storing a reagent or a material in a fluid communicable manner.

One or more embodiments of the present invention will now be described more fully with reference to the following examples. However, these examples are provided for illustrative purposes only and are not intended to limit the scope of the present invention.

### EXAMPLE 1

### Preparation of Nanopore Device And Analysis of Biomolecules Translocating Through Nanopores Using Asymmetric Electrolyte Concentration

### (1) Preparation of Nanopore Device

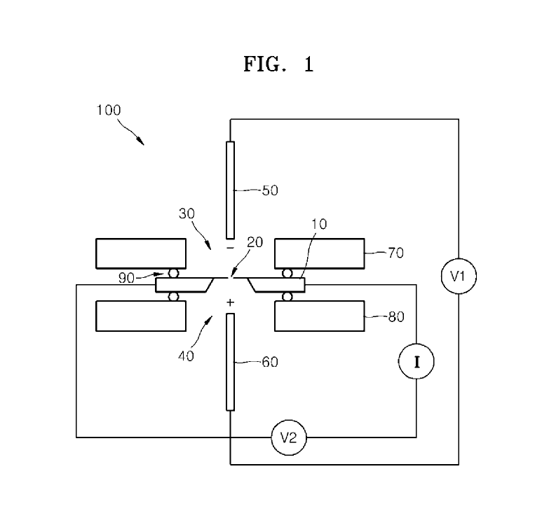

FIG. 1 is a schematic diagram of a device 100 for linearly translocating biomolecules via nanopores.

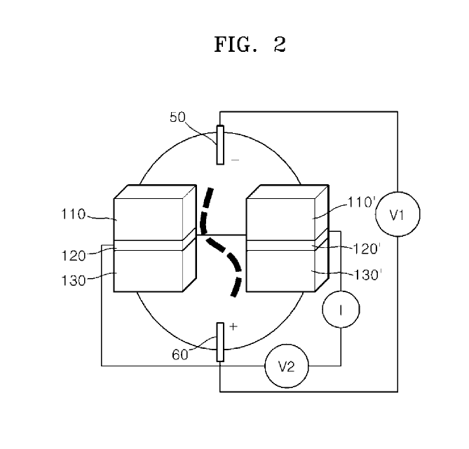

The device 100 includes: a cis chamber 30 for holding a liquid; a trans chamber 40 for holding a liquid; a substrate 10 including one or more nanopores 20 that are connected to the cis chamber 30 and the trans chamber 40 in a fluid communicable manner, wherein the nanopores 20 are formed to penetrate the substrate 10 in a thickness direction and have a first end and a second end opposite to the first end which are respectively connected to the cis chamber 30 and the trans chamber 40; and an electrode that is disposed to apply a voltage to a liquid that passes through the nanopores 20. The electrode disposed to apply a voltage to a liquid that passes through the nanopores 20 may include electrodes 50 and 60 respectively disposed at the first end side and the second end side opposite to the first end or, alternatively or simultaneously, a first electrode and a second electrode (shown in FIG. 2) which are disposed to be in contact with internal space of the nanopores 20 formed in the substrate by defining at least a portion of the wall of the nanopores 20. The first electrode and the second electrode may define at least a portion of the wall of the nanopores 20 by way of their terminal end portion.

In the solid substrate 10, the nanopore 20 may be formed in a silicon nitride (Si₃N₄) layer. The silicon nitride (Si₃N₄) layer may have a thickness of about 30 nm.

The device 100 may include the trans chamber 40 for holding a liquid and a pair of electrodes 50 and 60 which are units to translocate biomolecules via the nanopores 20 and/or electrical detection units. The cis and trans chambers 30 and 40 may hold liquids since upper and lower plates 70 and 80 thereof are respectively sealed by a sealing unit, for example, an O-ring 90.

FIG. 2 shows an enlarged portion which defines a nanopore 20 of a substrate of the FIG. 1.

Referring to FIG. 2, the substrate 10 includes an electrode pair of a first electrode 120 and a second electrode 120′ disposed thereon, wherein the electrode pair defines at least a portion of the walls of the nanopore 20 to be in contact with the internal space of the nanopore 20. The substrate 10 may have a stack structure in which first insulating materials 130 and 130′, the first electrode 120 and the second electrode 120′, and second insulating materials 110 and 110′ are sequentially stacked. The first insulating materials 130 and 130′ may be silicon nitride (SiN). The second insulating materials 110 and 110′ may be aluminum oxide (Al₂O₃). The first electrode 120 and the second electrode 120′ may include chromium, platinum, gold, copper, mercury, carbon nanotubes, graphene, or any combination thereof. The first electrode 120 and the second electrode 120′ may have a thickness in a range of 5 nm to 15 nm. The first insulating materials 130 and 130′ and the second insulating materials 110 and 110′ may respectively have a thickness in a range of 20 nm to 40 nm. As a result, the upper and lower surfaces of the first electrode 120 and the second electrode 120′ may be insulated from a first electrolytic solution and a second electrolytic solution respectively contained in the cis chamber 30 and the trans chamber 40. Referring to FIG. 2, the first electrode 120 and the second electrode 120′ are not disposed on the entire circumference of the cross-section of the nanopore 20 (e.g., if the cross-section is a circle), the circumference of the circle. Rather, the first electrode 120 and the second electrode 120′ are disposed not to be in contact with each other at the circumference of the cross-section. For example, the first electrode 120 and the second electrode 120′ may be aligned as an electrode pair facing the internal space of the nanopore 20 in a nanoribbon shape. Referring to a top view of the nanopore 20, the first electrode 120 and the second electrode 120′ may be disposed to constitute portions of the circumference of the nanopore 20 not to be in contact with each other.

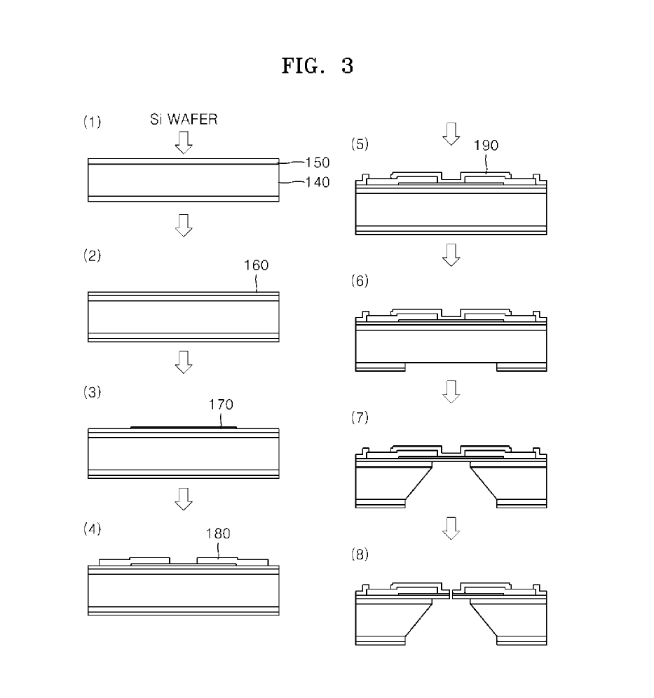

FIG. 3 is a diagram for describing a method of manufacturing the device 100 of FIG. 1. Referring to FIG. 3, first, as insulating layers, SiO₂ 150, is deposited on both sides of a silicon substrate 140 having a thickness of about 300 μm to a thickness of about 300 nm. The deposition is performed by thermal growth (1).

Then, low stress SiN thin films 160 for processing the nanopore 20 are formed on the insulating layers 150, respectively, by using a low-pressure chemical vapor deposition (LPCVD) (2).

Then, a first metal 170 that will function as an electrode of a nanogap is deposited thereon and patterned by using an e-beam lithography process. The first metal 170 is chromium (Cr) and has a thickness of about 10 nm and a width of about 50 nm. An etching of the lithography process was conducted by a lift-off process (3).

Then, a second electrode 180 that will be subjected to probing of a probe station or wire-bonding is prepared by depositing Au/Ti or Au/Cr, and at etching process is performed by a lift-off process as in the preparation of the first electrode. Au/Ti or Au/Cr may be deposited to a thickness of about 50 nm/10 nm (4).

Then, for the insulation of a metal electrode, an Al₂O₃ thin film 190 is deposited to a thickness of about 30 nm by using an atomic layer deposition (ALD) process for uniform insulation effects (5).

If the insulation of the electrode is completed, an etching process is performed on the rear surface of the Si substrate for processing the nanopore. The SiO₂ layer that is formed as an initial insulation layer and the low stress SiN thin film for processing the nanopore play roles as hardmasks (6).

Then, Si on the rear surface is removed by an Si etching process performed by using tetramethylammonium hydroxide (TMAH) or KOH. The SiO₂ thin film is removed by a buffered oxide etch (BOE) process, and the low stress SiN layer, the first electrode, and the Al₂O₃ insulating layer remain (7).

Then, by using a transmission electron microscope (TEM) equipment, a Cr electrode is partially removed to prepare a nanopore electrode (8).

### (2) Verification of Insulation Performance of Insulating Layer

Insulation performance of the Al₂O₃ insulating layer of the nanopore device prepared according to the above method was evaluated. Insufficient insulation performance of the insulating layer may cause leakage of current in a metal electrode to generate noise, so that a proper signal cannot be obtained. First, a 1 M KCl solution was dropped between nanopore electrodes. In this regard, the dropping was carefully performed such that the solution did not contact a pad portion of the second electrode. If the solution contacts the pad, the solution contacts the electrode regardless of the insulating layer so that insulating property cannot be obtained. Then, a probe tip of a probe station was connected to one of both sides of the second electrode, and a probe tip of the other side was dipped in a 1 M KCl solution. Current that flows between the probe tips was measured while applying a voltage to both ends of the electrode. As the insulating properties of the insulating layer are improved, current does not flow at a higher voltage. As a result of the measurement using the nanopore device prepared according to the above process, it was confirmed that current flowed when a voltage higher than 900 mV was applied thereto, so that the nanopore device could be used at a voltage less than 900 mV.

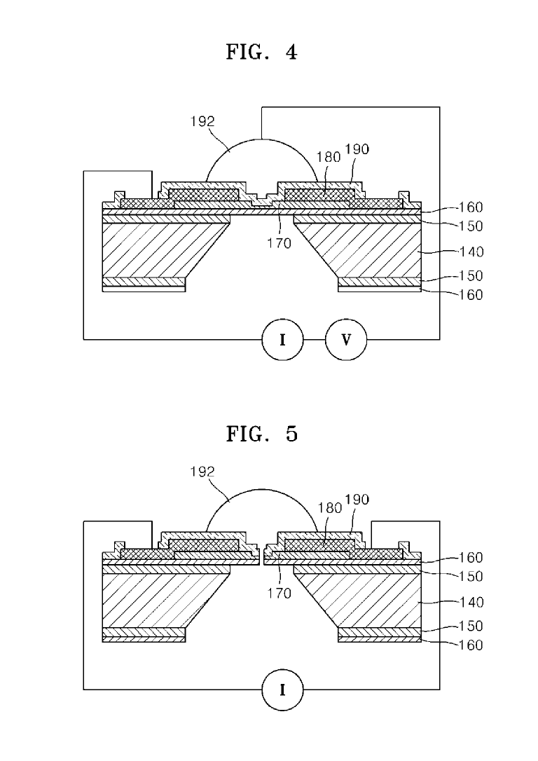

FIG. 4 is a schematic diagram illustrating a device for measuring leakage current of the insulating layer. Referring to FIG. 4, two electrodes were used to measure leakage current of the insulating layer. Thus, leakage current that occurs in the insulating layer between electrodes was only measured, but the current flowing in the nanopore was not measured. In FIG. 4, 1 M KCl drop 192 is disposed on the insulating layer, which is the Al₂O₃ thin film 190.

### (3) Changes of Noise And Power Spectrum With Respect To Concentration of Electrolyte

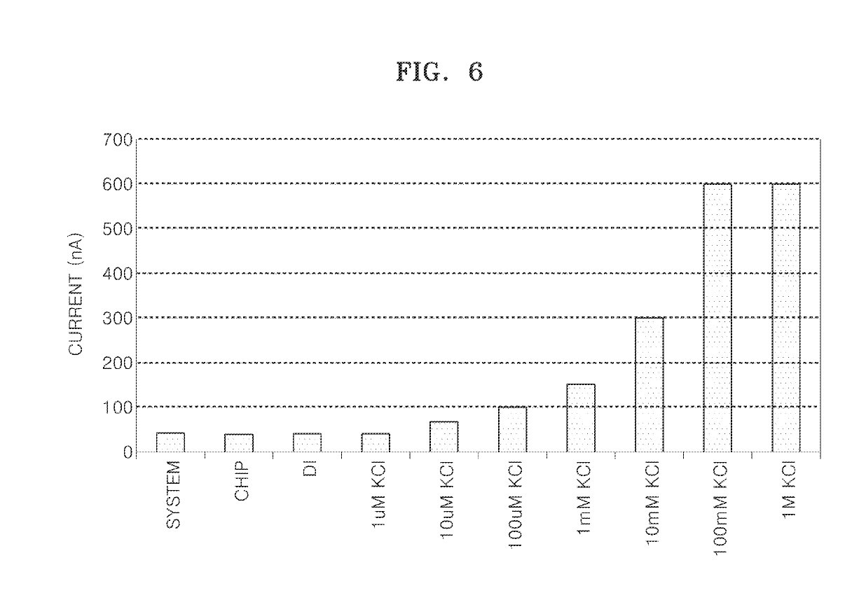

A relationship between the concentration of the electrolytic solution and noise was confirmed by using the nanopore device manufactured by using the method. 1 μM, 10 μM, 100 μM, 1 mM, 10 mM, 100 mM, and 1 M KCl solutions were prepared. Concentrations of each solution were determined by measuring electrical conductivity using a conductivity meter. The 1 M KCl solution showed about 141 mS/cm of electrical conductivity. As the concentration decreased, the electrical conductivity decreased. After the probe tips of the probe station were connected to both electrodes 180 (FIG. 5) of the nanopore device, noise generated at time domains of both electrodes was measured by using a p-Clamp equipment (FIG. 5). FIG. 5 is a schematic diagram illustrating a device for observing changes of noise and a power spectrum according to the concentration of electrolyte. Referring to FIG. 5, the internal space of the nanopore contacts a pair of metal electrodes 170, for example, Cr electrodes. The KCl drop 192 is disposed on the nanopore, and the internal space of the nanopore was filled with the KCl solution. The filling of the internal space of the nanopore with the KCl solution is considered to be caused by a capillary phenomenon, but is not limited thereto.

The noise of the probe station system before loading the measuring device of FIG. 5 was about 40 pA. After the measuring device was installed, the noise level was not considerably changed. Although the 1 μM KCl solution was dropped between the nanopore electrodes, the noise level was not changed. However, if a 100 μM KCl solution was dropped on the nanopore electrode, noise about 100 pA that was about twice greater than the previous level was measured. In a 10 mM KCl solution, noise was 350 pA, and in a more than 100 mM KCl solution, noise was more than 600 pA.

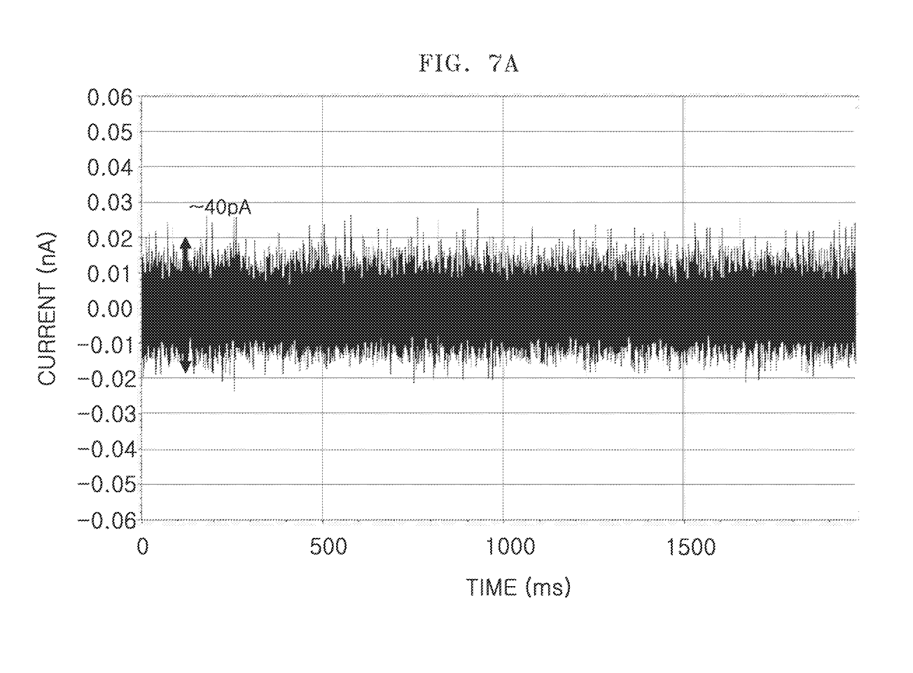

FIG. 6 is a graph illustrating noise measured according to the concentration of the electrolyte. In FIG. 6, the system is a probe station system, the chip is a measuring device, and DI is distilled water.

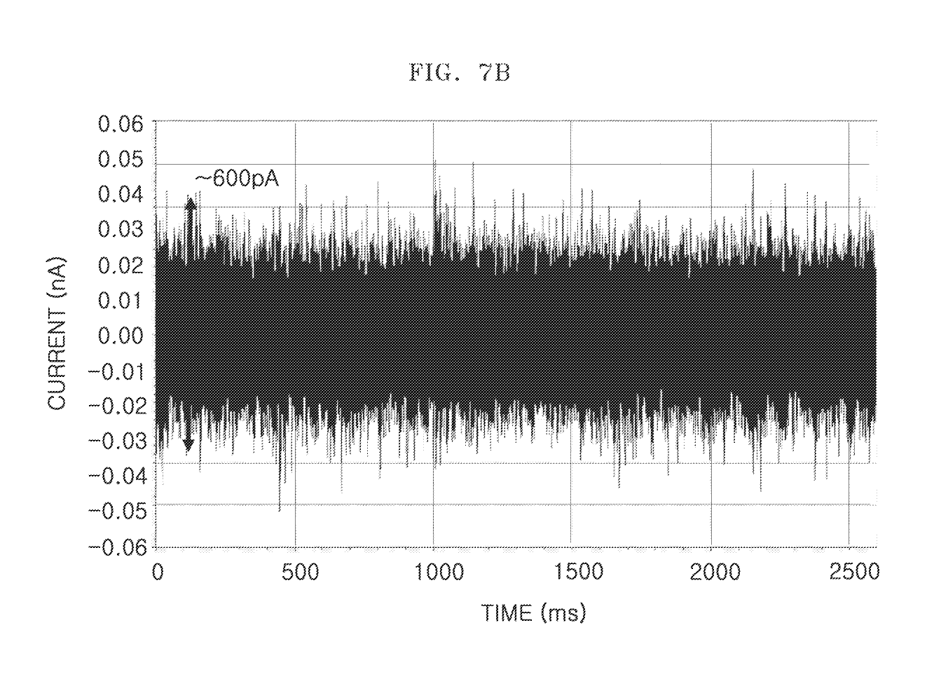

FIGS. 7A and 7B are diagrams illustrating noise of distilled water (7A) and 1 M KCl (7B). In FIG. 7A, the noise was about 40 pA, and in FIG. 7B, the noise was about 600 pA. In a power spectrum in which noise measured in the time domain was converted into a frequency domain, as the concentration of the KCl solution increased, noise increased in all frequency domains.

### (4) Observation of Translocation of DNA In Nanopore Device

Translocation of DNA was observed by using the nanopore device manufactured according to the method. The level of noise was observed by the experiment 1) when the 1 M KCl solution commonly used in general nanopore experiments was used. If the 1 M KCl solution was used in the upper and lower chambers of the nanopore device, desirable results could not be obtained due to high noise. The solution used in the lower chamber of the nanopore device was a 1 M KCl aqueous solution including 5 n/μL of λ-DNA. Since the degree of self-folding of DNA increases in a dilute solution, the possibility of passing through the nanopore decreases. Thus, the experiment could be conducted in an electrolyte having an appropriate concentration or higher. In the upper chamber, a 10 μM KCl aqueous solution was used in order to reduce the level of the noise. In addition, an Ag/AgCl electrode was disposed in the solution to generate an electric field to induce electrical translocation of DNA. A Cr electrode was prepared by connecting a probe tip with a pad of the second electrode so that current generated in the nanopore was measured (FIGS. 1 and 2).

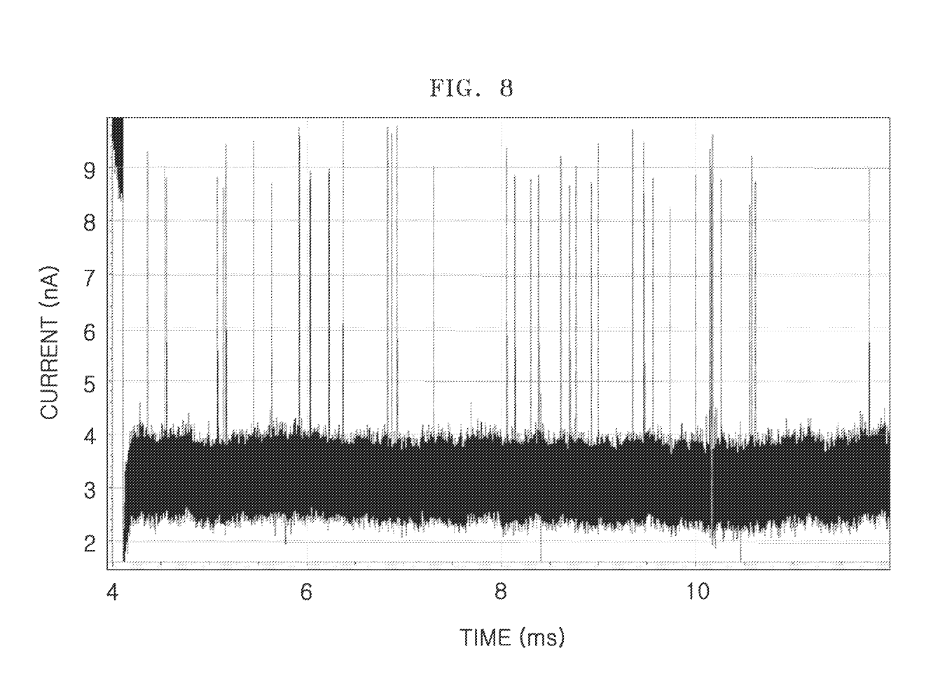

FIG. 8 is a graph illustrating current measured according to DNA transit time.

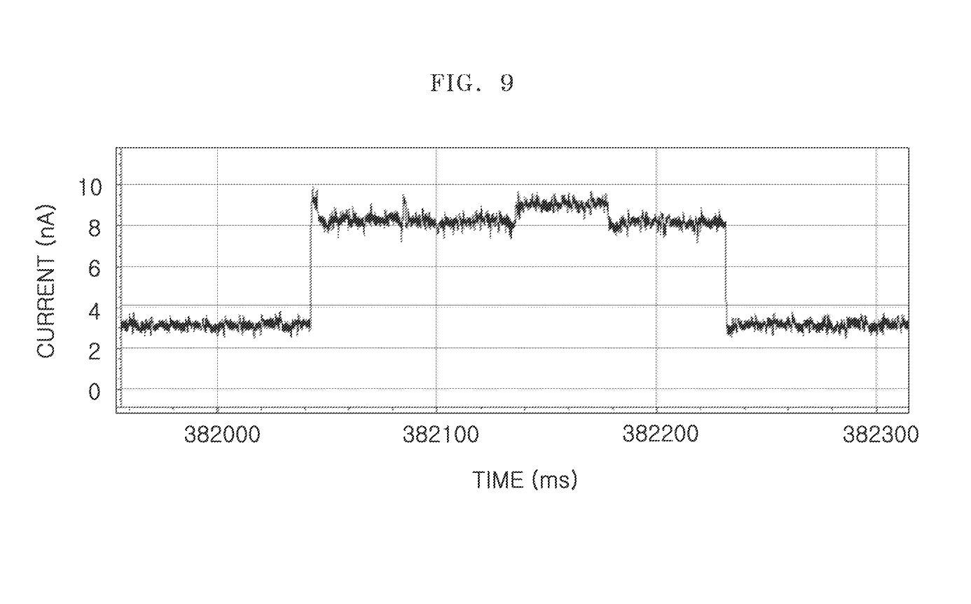

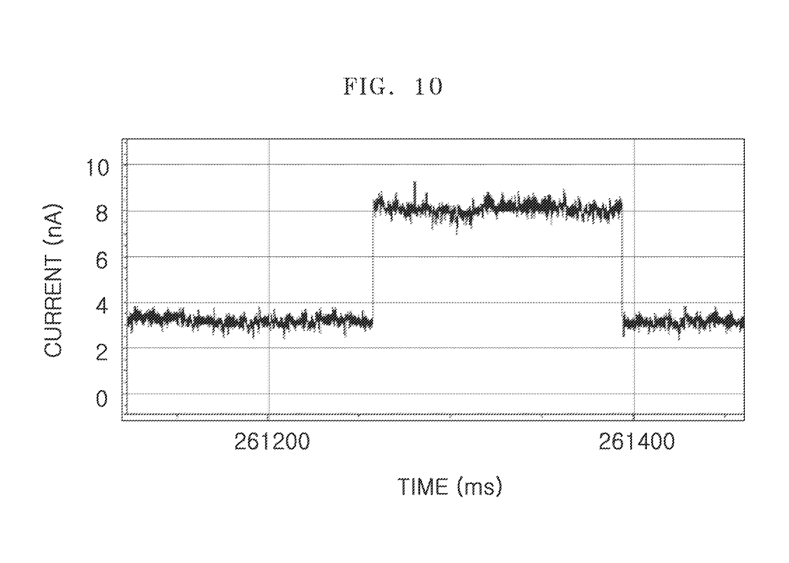

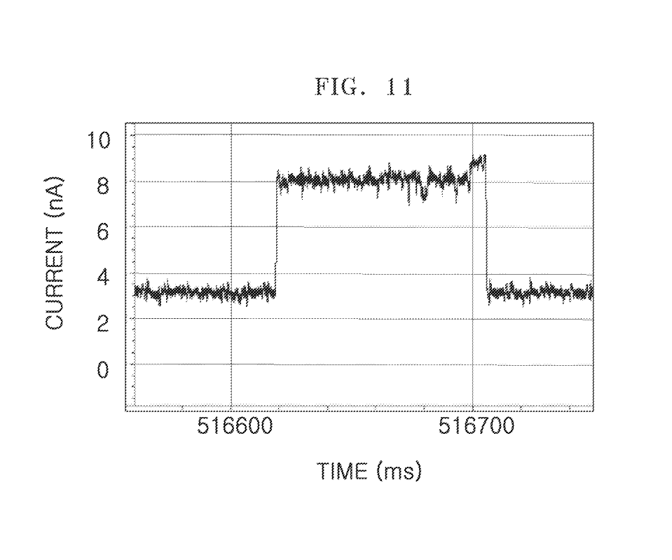

FIGS. 9 to 11 are graphs illustrating changes of current according to DNA transit time determined in FIG. 8. Referring to FIGS. 9 to 11, DNA transit time was in a range of about 80 ms to 180 ms. In addition, the current flowing in the nanopore according to the translocation of DNA was increased by about 5 nA.

As described above, according to a method of analyzing biomolecules in the biomolecule-containing first electrolytic solution, according to one or more embodiments of the present invention, the biomolecules contained in the first electrolytic solution may be efficiently analyzed. For example, a ratio of a signal to a noise may be increased by reducing the noise. Since a solution with a high electrolyte concentration may be used in the cis chamber, various samples may be analyzed. In addition, the translocation rate of the biomolecules may be reduced by the movement of water from the trans chamber to the cis chamber, and thus, an efficiency of the analysis may be increased.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of analyzing biomolecules, the method comprising:
providing a first electrolytic solution containing biomolecules to a cis chamber of a device that comprises:
a cis chamber for holding a liquid;
a trans chamber for holding a liquid;
a substrate comprising one or more nanopores that penetrate the substrate in a thickness direction, wherein the nanopores have a first end and a second end opposite to the first end which are in fluid communication with the cis chamber and the trans chamber, respectively; and
one or more electrodes positioned to apply a voltage to a liquid that passes through the one or more nanopores;
providing a second electrolytic solution to the trans chamber;
translocating the biomolecules from the cis chamber to the trans chamber; and
measuring an electric signal that is caused by the translocation of the biomolecules through the one or more nanopores,
wherein a ratio of a concentration of electrolyte in the first electrolytic solution to a concentration of electrolyte in the second electrolytic solution is equal to or greater than 10:1.

2. The method of claim 1, wherein the electrolyte comprises salts, acids, bases, or any combination thereof.

3. The method of claim 1, wherein the ratio of the concentration of the electrolyte of the first electrolytic solution to that of the second electrolytic solution is in a range of 10 to 100,000:1.

4. The method of claim 1, wherein the one or more electrodes comprises a pair of a first electrode and a second electrode disposed on the substrate, wherein the first electrode and the second electrode are disposed to be in contact with the internal space of the nanopore by defining at least a portion of the wall of the nanopore between the first and second electrodes.

5. The method of claim 4, wherein the first electrode and the second electrode are electrically connected to a power source, an electric signal measuring device, or both.

6. The method of claim 5, wherein the measuring is conducted by measuring a tunneling current between the biomolecule passing through the nanopore and the electrodes, or a blockade current by the biomolecule passing through nanopore.

7. The method of claim 1, wherein the electrode further comprises a third electrode disposed at a first end of the nanopore and a fourth electrode disposed at a second end of the nanopore.

8. The method of claim 7, wherein the third electrode and the fourth electrode are electrically connected to a power source, an electric signal measuring device, or both.

9. The method of claim 8, wherein the measuring is conducted by measuring a tunneling current between the biomolecule passing through the nanopore and the electrodes or a blockade current by the biomolecule passing through nanopore.

10. The method of claim 1, wherein a length of a cross-section of the nanopore is in a range of 1 nm to 100 nm.

11. The method of claim 10, wherein a length of a cross-section of the nanopore is in a range of 1 nm to 10 nm.

12. The method of claim 1, wherein the first electrolytic solution has a salt concentration of 1 mM to 1 M.

13. The method of claim 1, wherein the first electrolytic solution and the second electrolytic solution comprise the same type of salts.

14. The method of claim 1, wherein the salt comprises KCl, NaCl, LiCl, or any combination thereof.

15. The method of claim 1, further comprising determining a sequence of the biomolecule based on the measured electric signal.

16. The method of claim 1, wherein the biomolecule comprises DNA, RNA, or any combination thereof.

17. A system for analyzing biomolecules comprising:
a cis chamber containing a first electrolytic solution comprising biomolecules;
a trans chamber containing a second electrolytic solution;
a substrate comprising one or more nanopores that penetrate the substrate in a thickness direction and have a first end and a second end opposite to the first end which are in fluid communication with the cis chamber and the trans chamber, respectively; and
a pair of electrodes positioned to apply a voltage to a liquid that passes through the one or more nanopores,
wherein the pair of electrodes comprises a first electrode and a second electrode disposed on the substrate, wherein the first electrode and the second electrode define at least a portion of the wall of the one or more nanopores such that the first and second electrodes are in contact with the internal space of the one or more nanopores wherein a ratio of a concentration of electrolyte in the first electrolytic solution to that in the second electrolytic solution is equal to or greater than 10:1.

18. The system of claim 17, wherein the top and bottom surfaces of each of the first electrode and the second electrode are insulated from the first electrolytic solution and the second electrolytic solution by insulating layers.

19. The system of claim 18, wherein the insulating layer comprises silicon nitride, silicon oxide, aluminum oxide, hafnium oxide, or any combination thereof.

20. The system of claim 17, wherein the pair of the first electrode and second electrode are electrically connected to a power source, an electric signal measuring device, or both.

21. The system of claim 17, further comprising a third electrode disposed at the first end of the nanopore and a fourth electrode disposed at the second end of the nanopore.

22. A method of analyzing biomolecules, the method comprising:
introducing a biomolecule into a device comprising:
a cis chamber containing a first electrolytic solution;
a trans chamber containing a second electrolytic solution;
a substrate comprising one or more nanopores that penetrate the substrate in a thickness direction, wherein the nanopores have a first end and a second end opposite to the first end which are in fluid communication with the cis chamber and the trans chamber, respectively; and
one or more electrodes positioned to apply a voltage to a liquid that passes through the one or more nanopores;
translocating the biomolecules from the cis chamber to the trans chamber; and
measuring an electric signal that is caused by the translocation of the biomolecules through the one or more nanopores,
wherein a ratio of a concentration of electrolyte in the first electrolytic solution to a concentration of electrolyte in the second electrolytic solution is equal to or greater than 10:1.
